(19) United States
(12) Patent Application Publication
(10) Pub. No.: US 2007/0021786 A1
(43) Pub. Date: Jan. 25, 2007

(54) Selective nerve stimulation for the treatment of angina pectoris

(75) Inventors: Steven M. Parnis, Pearland, TX (US); Burke T. Barrett, Franklin, MA (US)

Correspondence Address: CYBERONICS, INC., LEGAL DEPARTMENT, 6TH FLOOR, 100 CYBERONICS BOULEVARD, HOUSTON, TX, 77058, US

(73) Assignee: CYBERONICS, INC.

(21) Appl. No.: 11/188,971

(22) Filed: Jul. 25, 2005

(51) Int. Cl. A61N 1/18

(52) U.S. Cl. 607/2

(57) ABSTRACT

A method is disclosed for electrically stimulating a cranial nerve, especially a vagus nerve, to treat or alleviate angina pectoris. Pain is lessened or prevented by application of predetermined therapeutic electrical signal to a selected location on the cranial nerve of a patient using an implanted neurostimulating device. Such method employs selective application of electrical signals to a predetermined location on the nerve to alter the activity of the nerve and cause dilation of a coronary artery in the patient, which in turn provides complete or partial relief of chest pain or deters the onset of such pain.

### BACKGROUND OF THE INVENTION

1. Field of the Invention

The present invention generally relates to methods and apparatus for electrically stimulating a cranial nerve, especially a vagus nerve, to treat or alleviate angina pectoris (tightening in the chest causing chest pain) or to deter the onset of an episode of such pain. More particularly, the invention pertains to such methods which apply stimulation to a predetermined location on the nerve to selectively produce dilation of a portion of a coronary artery, whereby angina is ameliorated or deterred.

2. Description of Related Art

Angina pectoris results from constriction of the coronary arteries of the heart and the consequential reduction of oxygen supply to the heart muscle. It is known that vagus nerve stimulation (VNS) can, under certain stimulation conditions, have the effect of slowing the heart rate. Reducing heart rate by VNS has been used in the past to reduce angina symptoms, since reduction of heart rate also reduces the heart muscle's demand for oxygenated blood and, by extension, the load on thecoronary arteries. Vagus nerve stimulation has also been used to slow the heart rate during open heart surgery, in lieu of a heart-lung bypass machine. U.S. Pat. No. 5,330,507 (Medtronic, Inc.) notes that early investigations of vagus nerve stimulation in the treatment of supraventricular arrhythmias, angina pectoris, and heart failure employing an implantable vagus nerve stimulator were reported at least as early as the 1960s.

Stimulation of the carotid sinus nerve was performed clinically in the 1960s and 1970s as a way to treat intractable angina pectoris. While those clinical results demonstrated relief of symptoms in a majority of cases, the mechanism of action was unknown or not well understood, and there were reports of serious drawbacks from the surgical implantation procedures or from anomalous induction of bradycardia (1-8).

The early investigators typically focused on controlling angina pectoris by regulating the patient's blood pressure through stimulation of the carotid sinus nerve. They observed that the effect of the application of carotid sinus nerve stimulation provided a safe means for initiating reflex vagus nerve activity which in turn effected a slowing in a patient's supraventricular tachycardia. Stimulation to the right or left vagus nerve, either directly or indirectly, had the effect of slowing tachycardia with the attendant risk of inducing bradyarrhythmia in the process. It has been said that the difficulty in determining the proper amplitudes, frequencies and durations of the electrical stimulation, coupled with continuing difficulty of chronic nerve stimulation, led to near abandonment of this therapy.

Previous investigations involving the application of electrical stimulation to vagus, carotid sinus and other nerves were typically effected using “cuff” type coated, bipolar electrodes, such as those disclosed in U.S. Pat. No. 3,421,511 (Medtronic, Inc.) surgically placed around the intact nerve or nerves. U.S. Pat. No. 4,573,481 (Huntington Institute of Applied Research) describes some of the problems associated with connecting electrodes to nerves, and discloses an open helical electrode design.

U.S. Pat. No. 5,330,515 (Cyberonics, Inc.) discloses a method and apparatus for blocking the sensation of pain through selective stimulation of the afferents of the vagus nerve to activate a descending anti-nociceptive pathway of the nerve tract. That method is not recommended in the case of true nociceptive pain because of its tendency to mask the early and ongoing warning signs of potentially serious disease or disorder, such as in patients with angina who may be experiencing ischemia and are at risk of myocardial infarction.

U.S. Pat. No. 5,707,400 (Cyberonics, Inc.) discloses a method of treating refractory hypertension by applying a stimulating electrical signal to the patient's vagus nerve. The signal is predetermined to modulate the electrical activity of the nerve and to reduce blood pressure.

U.S. Pat. No. 6,473,644 (Cyberonics, Inc.) discloses a method for treating patients suffering from heart failure. An electrical signal applied to a portion of the vagus nerve modulates the electrical activity of the nerve to adjust the ventricular rate of the patient's heart. In implementing that method, it is said to be important to stimulate the cardiac branches of the vagus nerve, since stimulation of the vagus nerve in the neck below the cardiac branches will not generally affect the heart rate.

U.S. Pat. No. 5,199,428 (Medtronic, Inc.) describes an implantable electrical nerve stimulator/pacemaker with ischemia detector for decreasing cardiac workload. Detection of ischemia, with or without attendant angina, and developing a stimulation trigger signal in response to an output signal indicative of a condition of ischemia for influencing the patient's blood pressure regulatory system through stimulation of the patients nerve system is discussed.

U.S. Pat. No. 6,073,048 (Medtronic, Inc.) describes baroreflex modulation with carotid sinus nerve stimulation for the treatment of heart failure. According to that method, the implantable system stimulates the patient's nervous system activating the baroreflex which in turn decreases sympathetic activity and increases parasympathetic activity.

In a study to determine a neurophysiological basis for cardiac pain referred to C₁-C₃ somatic dermatomes, it was suggested that vagal and/or sympathetic afferent activation of C₁-C₃ spinothalamic tract (STT) neurons may provide a neural mechanism for referred pain that originates in the heart or other visceral organs but is perceived in the neck and jaw regon (9).

Today, administration of beta blocking agents, nitrates and/or calcium antagonists are typical treatments for patients suffering from chronic stable angina pectoris. Treatment of unstable angina, which is associated with plaque and vessel obstruction, includes the placement of vascular stents, if indicated, and administration of beta blocking agents. As well-described in the scientific literature, beta blockage reduces the oxygen demand of the heart by lowering heart rate and blood pressure. Patients taking prescribed beta-blocking agents over a period of time often experience various undesirable side effects, however, including depression, bronchospasm and fatigue. Even with these side effects, beta blocker therapy can be highly beneficial to those patients who can tolerate the side effects. Conventional treatment for acute angina pectoris typically includes sublingual administration of nitroglycerin. Preventing or reversing coronary artery constriction and the associated pain by using effective vagus nerve stimulation instead of a problematic drug would be a welcome therapy option. An implantable nerve stimulation system might benefit patients suffering from acute or chronic angina by alleviating the pain and avoiding the drawbacks of relying solely on drugs to treat angina.

### BRIEF SUMMARY OF THE PREFERRED EMBODIMENTS

A new, improved way to treat angina pectoris (“angina”) is provided which focuses specifically on dilating a coronary artery by selective stimulation of a cranial nerve, rather than on slowing the heart rate orreducing blood pressure. The new method makes it possible to avoid some of the undesirable side effects that have characterized known treatments, such as drug therapy. More specifically, cranial nerve stimulation, preferably left and/or right vagus nerve stimulation, is applied to selectively and controllably modulate the nerve's electrical activity so as to alleviate or deter the onsetof pain associated with angina pectoris.

Accordingly, there is provided a method of treating angina pectoris in a patient in need thereof The method generally comprises coupling an electrode to a cranial nerve of the patient at a selected site on the nerve, and then applying at least one therapeutic electrical signal to the electrode. The electrical signal is provided so as to dilate at least a portion of a coronary artery of the patient, thereby alleviating pain associated with angina pectoris and/or deterring the occurrence of an episode of acute angina pectoris. In some embodiments, the coupling step comprises coupling the electrode to a vagus nerve of the patient, e.g., the left vagus nerve of the patient. In certain embodiments, the method includes selecting an electrical signal that is capable of dilating the artery, or at least a portion ofthe artery, without significantly slowing the heart rate ofthe patient.

In certain of the above-described embodiments the method includes (a) providing an electrical pulse generator capable of generating a pulsed electrical signal; (b) providing at least one electrode; (c) implanting the electrical pulse generator in the patient's body; (d) surgically coupling at least one electrode directly to a vagus nerve of the patient at a selected site on the nerve that is capable of causing dilation of at least a portion of a coronary artery of the patient when electrical stimulation is applied to the site; (e) coupling the electrical pulse generator to the electrode(s); (f) generating a pulsed electrical signal using the electrical pulse generator; and (g) applying the pulsed electrical signal to the vagus nerve using the electrode(s) such that at least a portion of a coronary artery dilates and thereby at least partially relieves a symptom of the patients angina.

Certain embodiments of the preceding method include programming the electrical pulse generator to define a pulsed electrical signal by a plurality of predetermined parameters, including a current magnitude, a pulse frequency, and a pulse width, wherein the signal thus defined causes dilation of at least a portion of a coronary artery of the patient when the pulsed electrical signal is applied to the electrode(s).

Where cranial nerve stimulation is provided based solely on programmed off-times and on-times (which may also be used to provide stimulation according to circadian rhythms), the stimulation is referred to as passive, inactive, or non-feedback stimulation. In contrast, stimulation may be triggered by one or more feedback loops according to changes within the body or mind of the patient. Such stimulation is referred to as active or feedback loop stimulation. Both passive and active stimulation may be used n embodiments ofthe present invention.

In certain embodiments, an above-described method also includes detecting at least one indicator of angina pectoris in the patient; and then initiating the application of the electrical signal to the vagus nerve in response to the detection ofthe at least one indicator.

The detecting step may include, for example, providing an implantable sensor that is capable of sensing a predetermined symptom or indicator of angina pectoris; implanting the sensor in the patient's body at a predetermined sensing site; coupling the sensor to the electrical pulse generator; and detecting a symptom or indicator of angina pectoris with the implanted sensor. For example, a patient experiencing chest pain may place a magnet over the skin adjacent to the implanted electrical pulse generator to begin application ofthe therapeutic pulsed electrical signal.

In certain embodiments of an above-described method, the therapeutic electrical signals comprise a first (acute stage) therapeutic electrical signal effective for treating acute angina symptoms, e.g., chest pain, and also comprises a second (chronic stage) therapeutic electrical signal that comprises a lower level of stimulation than the first stage, as effective for treating chronic angina. In some embodiments the second signal continues after the first signal ceases, and in some embodiments the first signal is applied without ceasing application of the second signal. For instance, in the case of a patient suffering from chronic angina pectoris, the application of the second therapeutic electrical signal is effective to maintain sufficient dilation of at least a portion of the artery to deter or avoid onset of angina attacks. The first signal, having a higher level of stimulation, is more effective for alleviating pain associated with an acute episode of angina.

In certain embodiments of any of the above-described methods, the electrode is positioned on the main branch of the left or right vagus nerve in the neck/upper chest below the cardiac branches.

Certain embodiments of the above-described methods further include administering one or more pharmaceutical agents, e.g., beta blockers, nitrates, and/or calcium antagonists, to the patient to further alleviate angina pectoris or deter the onset of angina pectoris.

Also provided in accordance with certain embodiments of the present invention is a method of selectively dilating a coronary artery in a patient suffering from angina pectoris. The method generally comprises (a) coupling an electrode to a cranial nerve of the patient at a selected site on the nerve; and (b) applying at least one therapeutic electrical signal to the electrode effective to dilate at least a portion of the artery and thereby increase blood flow to the patient's heart such that the increased arterial blood flow alleviates angina pectoris or deters the onset of acute angina pectoris in the patient.

In certain embodiments, the foregoing method comprises selecting a site on the nerve that has been determined to be a coronary artery-dilation effective site, wherein when the effective site is stimulated, a portion of a coronary artery is caused to be dilated in response to such stimulation. In certain embodiments, the cranial nerve is the vagus nerve of the patient and the selected site is an area on the nerve below the cardiac branches of the vagus nerve. In certain preferred embodiments, the patient's heart rate is not caused to decrease significantly (i.e., no more than about 20%, preferably no more than about 10%) as a result of the application of the therapeutic electrical signal. These and other embodiments, features and advantages will be apparent from the following description and drawings.

### BRIEF DESCRIPTION OF THE DRAWINGS

FIG. 1 is a simplified partial side view of a patient (shown in phantom outline) illustrating one embodiment of a neurostimulator placement configuration for applying an electrical signal to the patient's right vagus nerve below the cardiac branches, in accordance with an embodiment of a treatment regimen of the present invention.

FIG. 2 is a front view of an implantable electrical pulse generator as employed in the configuration of FIG. 1.

FIG. 3 illustrates a lead and bipolar electrode assembly for attachment to a nerve, according to the configuration of FIG. 1.

FIGS. 4A-D are simplified partial front views of a patient (shown in phantom outline) illustrating alternative neurostimulator placement configurations for applying an electrical signal to the patient's left and/or right vagus nerve at a near-diaphragmatic location, in accordance with certain embodiments of the invention. FIG. 4A depicts a single electrical pulse generator and bilateral sub-diaphragmatic vagus nerve treatment configuration. FIG. 4A also depicts a single electrical pulse generator and bilateral supra-diaphragmatic vagus nerve treatment configuration (dashed lines). FIG. 4B depicts two electrical pulse generators with a subdiaphragmatic bilateral vagus nerve treatment configuration. FIG. 4C depicts a single pulse generator and a subdiaphragmatic right vagus nerve treatment configuration. FIG. 4D illustrates an embodiment like FIG. 4C except that the electrode is not directly coupled to the vagus nerve, to provide indirect electrical modulation ofthe nerve.

FIG. 5 is a simplified representation of a programmed output signal waveform as delivered to a sympathetic nerve, in accordance with certain methods of the present invention.

### DETAILED DESCRIPTION OF THE PREFERRED EMBODIMENTS

Definitions.

The phrases “alleviation of angina pectoris,” “relief of pain,” and similar phrases refer to and mean any of the following complete cessation of chronic or acute pain of angina pectoris; at least partial reduction in chronic or acute angina pectoris pain; and/or prevention or deterrence of the onset of an episode of acute angina pectoris.

“Significant slowing of heart rate” refers to a clinically significant slowing of the heart rate. For example, a rate change of more than about 20% would be significant for the purposes of the present disclosure.

“Acute angina” refers to a sudden onset of angina which usually resolves with drug, surgical, or deuce intervention

“Chronic angina” or “recurring angina” refers to angina which does not resolve in the short term, despite intervention. Chronic angina may be of a stable or unstable type.

“Stable angina” refers to angina which occurs only in response to a known amount of exercise or other stress, such as emotional stress.

“Unstable angina” refers to chest pain in which the intensity, frequency or duration of episodes is changed and can no longer be predicted. Episodes of pain are precipitated by less exercise or are of longer duration than with stable angina. For example, a first onset episode of pain is acute angina and is also considered unstable. Unstable angina may also refer to chronic, or recurrent angina, in which angina occurs at rest. This may signal an impending heart attack (myocardial infarction).

Prinzmetal's angina refers to again that occurs at rest, when sleeping or when exposed to cold temperatures. In such cases, the symptoms are caused by decreased blood flow to the heart muscle or from spasm of the coronary artery.

Electrical stimulation signals of various types may be used in the present invention. Typical cranial nerve stimulation techniques involve providing a pulsed electrical signal in a series of discrete pulse bursts. A pulsed electrical signal is one in which flow of current during an on-time period is separated by short periods (typically milliseconds or seconds) of no current flow. A non-pulsed signal, as used herein, refers to a signal in which a current is always being delivered during the on-time. Pulses within a pulse burst are typically defined by a plurality of programmable parameters including a current magnitude (usually expressed in milliamps), a pulse width (usually expressed in microseconds or milliseconds), and a frequency (usually expressed in Hz). Pulse bursts are usually delivered according to the programmed pulse characteristics during a programmed on-time interval, and bursts are usually separated by defined off-time periods, in which no stimulation signal is provided. Although the invention is described with respect to pulsed electrical signals, non-pulsed signals may also be used. It should be noted that non-pulsed signals may be delivered according to a programmed or random on-time and off-time; however, unless the on-time periods have breaks in current flow within each on-time period, the signal remains a non-pulsed signal as used herein

A continuous signal, as used herein, refers to an electrical signal without a distinct on-time and off-time. Accordingly, convention neurostimulation techniques are generally non-continuous signals. A continuous signal may be delivered as either a pulsed signal having a constant or random time period between pulses, as a non-pulsed signal with a constant or random time between stimulation signals, or as a purely continuous signal with no break in current flow at all (although other parameters, such as current magnitude and polarity, may vary within the signal).

“Including” and “comprising” are each used in an open-ended fashion, and thus should be interpreted to mean “including, but not limited to.”

“Couple” and “couples” are each intended to mean either an indirect or direct electrical connection. Thus, if a first device couples to a second device, that connection may be through a direct electrical connection, or through an indirect electrical connection via other devices and connections.

The inventors propose using cranial nerve stimulation to relieve angina pain by dilating at least a portion of a coronary artery, in contrast to prior art approaches that involve lowering the heart rate. Accordingly, the present invention may comprise coupling an electrode to a cranial nerve of a mammal at a certain site or region of the nerve, and then applying a therapeutic electrical signal to the electrode that is effective to produce dilation of at least a portion of a coronary artery of the mammal without a substantial lowering of heart rate. According to preferred embodiments, the degree of dilation is sufficient to provide an increase in the supply of oxygenated blood to the heart to alleviate or lessen an existing episode of angina, and/or to deter or prevent a recurrence of angina pectoris due to arterial constriction.

It is now proposed that therapeutic stimulation of selected sites or regions of the right, left or both right and left vagus nerve(s) will be effective in reducing or preventing angina pectoris, or chest pain associated with myocardial ischemia. Without wishing to be restricted to a single theory to explain the mechanism by which this result is achieved, it is presently thought that afferent stimulation of the vagus nerve causes a baroreceptor response terminating in the nucleus tractus solitarius (NTS) in the brain. In the NTS, neurons excite vasopressor cells of the dorsal vagal nucleus and nucleus ambiguus and inhibit vasopressor neurons of the rostral ventrolateral medulla. As a result, the arterial vascular bed is dilated and the vasoconstricting effect leading to angina is abated. The therapeutic vagus nerve stimulation causes vasodilation of coronary arteries, resulting in a decrease in blood pressure and lessening the work load required of the heart. Coronary blood flow also increases as a result of coronary artery dilation.

Although the preferred embodiments provide for stimulation of the 10th cranial nerve, also known as the vagus nerve, it is expected that other cranial nerves may be stimulated instead of, or in addition to the left and/or right vagus nerve, to produce at least some degree of relief of angina. For instance, the trigeminal nerve (5th cranial nerve) and the glossopharyngeal nerve (9th cranial nerve) may be stimulated at effective locations on the nerve. For example, in the case of the vagus nerve, the electrode is preferably positioned on the main branch of the left or right vagus nerve in the neck, below the cardiac branches.

A basic treatment and control system for use in the present invention comprises a neurostimulator including an electrical pulse generator, leads, electrodes and, optionally, sensors. Also included are external electronics for calibration, programming, and periodic adjustment of parameters by the attending physician according to the needs of the particular patient, and for monitoring the implanted device operation, through telemetry. Preferably, the electrical pulse generator is implemented or programmed to be activated passively, either for continuous or, more preferably, non-continuous stimulation, with regular off-time intervals of that may optionally be linked to the patient's circadian cycle. Alternatively, the device may be automatically triggered (i.e., active stimulation) to deliver the prescribed therapy in response to sensing a predetermined decrease in blood flow in a coronary artery, or another physiological indicator, or it may be manually triggered by the patient for a sustained period of stimulation at specified times, such as during periods of increased physical exertion or an increased perceived level of pain.

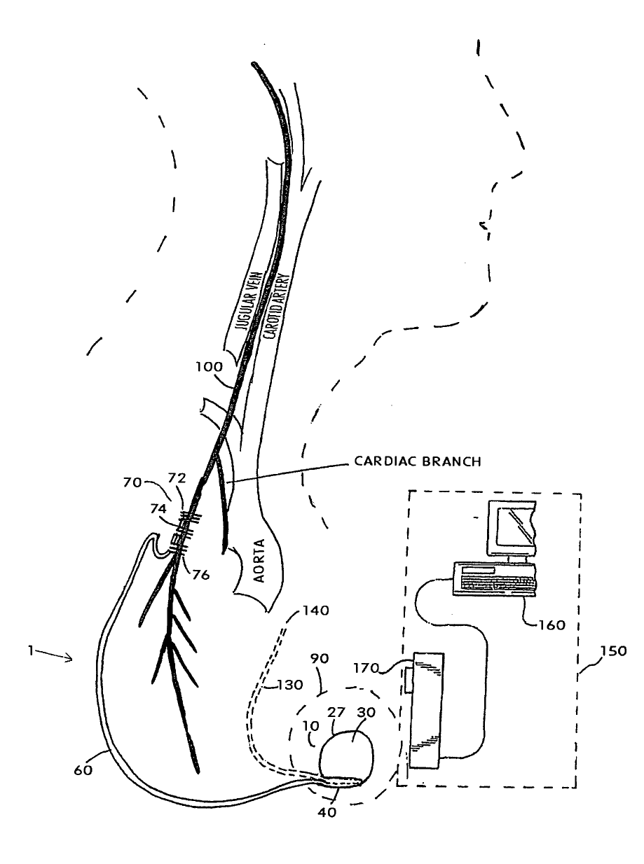

FIG. 1 illustrates a representative neurostimulation system (neurostimulator) 1 as employed for selectively stimulating a cranial nerve, in this embodiment the left vagus nerve 100, of a patient. A suitable neurostimulator is described in U.S. Pat. No. 5,154,172 (Cyberonics, Inc) and the NeuroCybernetic Prosthesis or NCP™ device is available from Cyberonics, Inc., Houston, Tex., U.S.A. Alternatively, other suitable neurostimulators may be employed. An exemplary neurostimulator generally includes an electrical pulse generator 10, at least one lead assembly (lead) 60 and an external programming system 150. Parameters defining the electrical signal generated by the neurostimulator 1 and delivered to the nerve 100 are preferably programmable over a range of values to allow a physician to tailor the stimulation provided to the patient, preferably by means of an external programmer 160 in a conventional manner for implantable electrical medical devices.

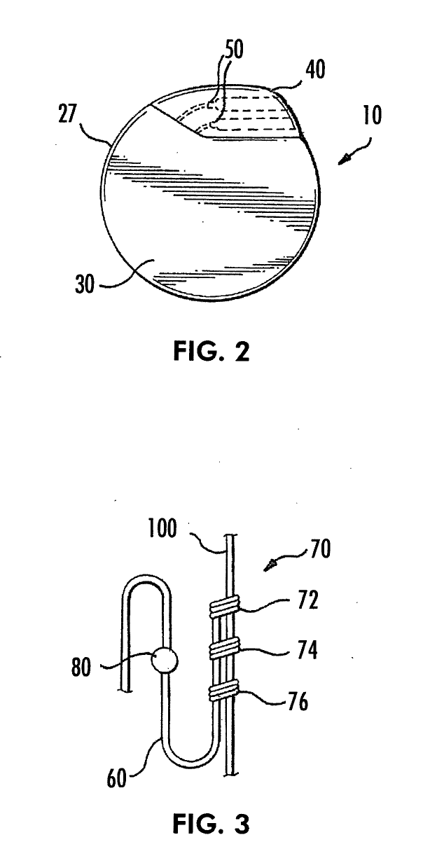

Referring now to FIG. 2, an implantable electrical pulse generator 10 is provided with a main body 30 comprising a case or shell 27 with a header 40 having at least one electrical connector 50 for connecting, respectively, to at least one lead 60. The electrical pulse generator preferably comprises a transmitter/receiver (not shown) for communication with external programming system 150.

FIG. 3 illustrates an electrode assembly 70 suitable for use as shown in FIG. 1. Electrode assembly 70 preferably comprises a bipolar stimulating electrode pair 72, 74 as described in U.S. Pat. No. 4,573,481 (Bullara). The electrode pair 72, 74 is preferably coupled to the distal end of an insulated electrically conductive lead assembly 60, which preferably comprises a pair of lead wires (one wire for each electrode of an electrode pair). Each lead wire in lead assembly 60 is preferably removably attached at its proximal end to a connector 50 on case 27 of electrical pulse generator 10 (FIG. 2). The preferred bipolar electrode assembly has an open helical design that is self-sizing and flexible, and thus minimizes trauma to the nerve while allowing body fluid interchange with the nerve. In one example, the electrode assembly 70 comprises two electrode ribbons (not shown), of a conductive material such as platinum, iridium, platinum-iridium alloys, and/or oxides of the foregoing. The electrode ribbons preferably are individually bonded to an inside surface of an elastomeric body portion of the two electrodes 72 and 74, which may comprise two spiral loops of a three-loop helical assembly. The elastomeric body portion of each loop is preferably composed of silicone rubber, and the third loop 76 (which typically has no electrode) acts as the anchoring tether for the electrode assembly 70. Notwithstanding the electrode configuration illustrated in FIG. 3, it will be appreciated by persons of skill in the art that a variety of electrodes, e.g., cuff electrodes, may be used in the present invention.

Lead assembly 60 also comprises two distinct lead wires or a coaxial cable whose two conductive elements are respectively coupled to one of the electrodes 72 and 74. One suitable method of coupling the lead wires or cable to the electrodes comprises a spacer assembly such as that disclosed in U.S. Pat. No. 5,531,778 (Cyberonics, Inc.), although other known coupling techniques may be used.

Referring again to FIG. 1, external programming system 150 is preferably capable of wireless (e.g., radio frequency) communication with the electrical pulse generator 10, and comprises a computer 160 and a wand 170 having an RF transmitter and receiver. Computer 160 preferably comprises a handheld computer operable by a healthcare provider. Wand 170 is capable of communicating with a receiver and transmitter in pulse generator 10, and may be used to receive data from or transmit data to the electrical pulse generator 10.

A variation of the above-described treatment configuration additionally provides for receiving and utilizing feedback from a body parameter to the electrical pulse generator. As shown in dashed lines in FIG. 1, the assembly may further include a sensing lead 130 (dashed lines) coupled at a proximal end to a connector 50 in header 40, similar to the stimulation lead and electrode assembly 60, 70. A sensor 140 is coupled to the distal end of sensing lead 130 (also shown in dashed lines). Sensor 140 may comprise an accelerometer, for example, capable of sensing blood flow rate in a coronary artery, or a pressure sensor for sensing blood pressure. Alternatively, a sensor 140 capable of detecting another suitable physiological parameter that is determined to be indicative of angina may be used instead.

Method of Reducing or Alleviating Angina Pectoris

A new treatment for angina pectoris generally includes implanting an electrical pulse generator and one or more electrode assemblies of a neurostimulator system into the patient to generate an electrical signal, preferably configured as a sequence of pulses in which the electrical and timing parameters have programmable values. These parameter values are selected by the attending physician to be within ranges appropriate for the treatment. As an example, the pulsed electrical signal is applied to a cranial nerve of the patient through an electrode set of a lead implanted on the nerve at a preselected site. Where the cranial nerve stimulated is a vagus nerve, the electrodes are preferably coupled to the nerve at a location in the patient's lower neck/upper chest area, below the cardiac branches. The electrical signal is preferably defined so as to modulate the electrical activity of the nerve and release one or more neurotransmitters to specifically cause a constricted artery to dilate or to deter constriction of the artery, without significantly lowering the heart rate. Neurotransmitters are classified as either excitatory or inhibitory. While electrical modulating signals may be selected to stimulate or inhibit, for purposes of this disclosure, an electrical signal of either type may be referred to as an “electrical stimulation signal,” or as “stimulating” the nerve in this specification. The specific stimulating signal pattern used to achieve a desired effect of the vagus nerve modulation for a prescribed treatment may be selected based on various factors, including the individual patient, the specific nature of any underlying cardiovascular disorder which may contribute to the patient's angina pectoris, and the nerve fiber types to be activated. The stimulation strategy may also depend on factors such as whether a symptom or indicator of the patient's angina pectoris can be sensed to activate the neurostimulator in a feedback stimulation regime, or a physiologic parameter can be detected to trigger the stimulation, and whether a refractory period after the stimulation interval allows the benefits of the nerve activity modulation to persist.

Referring again to FIG. 1, in a representative procedure for stimulating a patient's vagus nerve to produce dilation of a coronary artery and relief from angina, the above-described neurostimulator system 1 is employed. The electrical pulse generator 10 is implanted in the patient's chest in a pocket or cavity formed by the implanting surgeon below the skin (indicated by a dashed line 90), at a suitable location as determined by the surgeon. For example, placement may be similar to a customary implantation procedure for a heart pacemaker (which also comprises an electrical pulse generator). Alternatively, the electrical pulse generator 10 may be implanted in the patient's abdominal region via a left laparotomy incision. In the embodiment illustrated in FIG. 1, the electrode assembly 70 is surgically coupled to a vagus nerve 100 in the patient's lower neck-upper chest area (see also FIG. 3) at a selected site or region that has been determined be effective for producing a responsive dilatory effect on a coronary artery, or portion thereof, when effectively stimulated. The two electrodes 72, 74 are preferably wrapped about the vagus nerve, and the electrode assembly 70 is preferably secured to the nerve 100 by a spiral anchoring tether 76 (FIG. 3) such as that disclosed in U.S. Pat. No. 4,979,511 (Cyberonics, Inc.). The electrode assembly preferably conforms to the shape of the nerve, providing a low stimulation threshold by allowing a larger stimulation contact area. Alternatively, another suitable electrode design could be used. Lead assembly 60 is secured, while retaining the ability to flex with movement of the chest and neck, by a suture connection 80 to nearby tissue (FIG. 3). While the electrodes 72, 74 of electrode assembly 70 are shown in the preferred embodiment of directly contacting the vagus nerve 100, it is to be understood that alternative electrode placements are also contemplated in which the electrode does not directly contact the nerve, provided that the electrode is nevertheless electrically coupled to the vagus nerve 100 (or other cranial nerve).

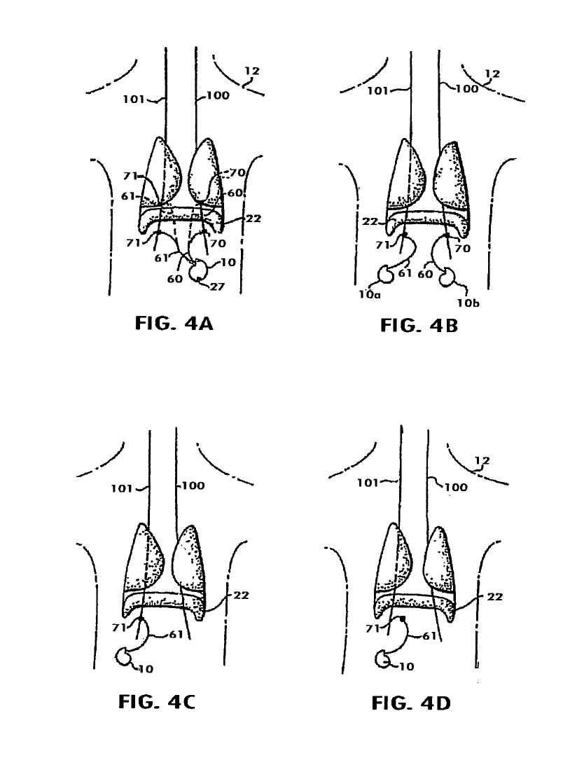

Although FIG. 1 illustrates a preferred configuration for stimulating a main vagus nerve at a site that is below the cardiac branches in the lower neck/upper chest area. Stimulation may be applied to the left or the right vagus nerve (or both) and the electrodes may alternatively be coupled to the nerve in the diaphragm region instead of the neck/chest area. In those instances, lead and electrode assemblies substantially like those described above may be coupled to the same or a different generator. For example, referring to FIG. 4A, for applying direct bilateral stimulation of the left and right vagus nerves, a first electrode assembly 70 (comprising electrodes 72, 74) at the distal end of lead assembly 60 is coupled to the left vagus nerve 100 at a location below the diaphragm 22. The proximal end of the lead 60 is electrically connected to the electrical pulse generator 10. This configuration includes a second electrode assembly 71, which may be substantially identical to assembly 70 previously described, and a second lead assembly 61, which may be identical to lead assembly 60 previously described. The second electrode assembly 71 is coupled to the right vagus nerve 101 at a sub-diaphragmatic location, and the proximal end of lead 61 is likewise electrically connected to neurostimulator 10.

FIG. 4A also depicts an alternative configuration, in which the electrode assemblies 70, 71 are coupled to the left and right vagus nerves, respectively above the diaphragm (i.e., supra-diaphragmatically), as shown in phantom lining Both supra-diaphragmatic and sub-diaphragmatic locations are encompassed within the term “near-diaphragmatic,” and where the latter term is used, either location is intended.

As illustrated in FIG. 4B, each lead/electrode assembly may be coupled to a separate electrical pulse generator 10a, 10b for stimulating the left and right vagus nerve branches, in contradistinction to the use of a single electrical pulse generator 10 as shown in FIGS. 1 and 4A. Use of multiple generators may extend the time between replacements ofthe generators by reducing the power drain for bilateral stimulation. In FIG. 4C, a system is shown for the direct, unilateral stimulation of the right vagus nerve 101, with an electrode assembly 71 attached to the vagus nerve 101 at the distal end of lead 61, which is coupled at its proximal end to electricalpulse generator 10. Finally, in FIG. 4D, a system is provided with an electrode 71 that is not in direct contact with the right vagus nerve 101, but which nevertheless retains an electrical coupling to the nerve that is sufficient, which energized by the electrical pulses from the pulse generator 10, to generate therapeutic action potentials on the nerve 101.

It will be understood that the overall device generally is required to be approved or sanctioned by government authority for marketing as a medical device implantable in a patient together with electrode means to treat the angina pectoris and/or to specifically dilate a portion of a coronary artery by selective stimulation of a cranial nerve or nerves (e.g., the vagus nerve) of the patient. Where the stimulation signal is applied to the vagus nerve, the treatment is performed using a predetermined sequence of electrical pulses generated by the electrical pulse generator 10 and applied to the left and/or right vagus nerve at a neck/upper chest, near-diaphragmatic, or other location on the vagus nerve that is below the vagus nerve cardiac branches, for dilating a portion of a coronary artery without significantly slowing the heart rate, and thereby causing alleviation of angina or deterrence of onset of angina. Other cranial nerves will typically involve coupling the electrode at a location in the patient's head or neck, provided that the selected location is identified as an effector site for causing dilation of a portion of a coronary artery and relieving angina, as described above.

Referring again to the representative treatment configuration illustrated in FIG. 1, neurostimulator 10 generates an electrical signal, preferably in the form of electrical pulses according to one or more programmed parameters for stimulation of a vagus nerve. Where non-continuous stimulation is provided, the stimulation parameters typically include pulse current magnitude, pulse width, frequency, and on-time or off-time. A table of ranges for each of these stimulation parameters is provided in Table 1. Parameters, such as current magnitude, may be adjusted accordingly until an absence of undesirableeffects such as significant lowering of the heart rate is observed, and such that the patient does not experience pain associated with the stimulation, with a suitable safety margin provided. While pain thresholds can approach 6.0 milliamps (mA), current more typically ranges from 0.5 to 3.0 mA, and in some cases pain thresholds may be significantly lower. In one exemplary treatment regime, a current of 1.5 mA is used. Tolerable and/or efficacious parameters may change with time over a course of days after implantation, and may be adjusted as necessary to maintain an effective regimen. Preferably, effective signal parameters are selected which cause a decrease in the heart rate of no more than 20% and preferably no more than 10%.

TABLE 1

| Parameter | Range |
|---|---|
| Output Current | 0.1-6.0 mA |
| Pulse Width | 10-1500 μsec |
| Frequency | 0.5-250 Hz |
| On-time | 1 sec-unlimited |
| Off-time | 0 sec-unlimited |
| Frequency Sweep | 10-100 Hz |
| Random Frequency | 10-100 Hz |

In non-continuous forms of stimulation, on-time and off-time parameters may be used to define an intermittent pattern in which a repeating series of pulses is generated for stimulating the nerve during the on-time (such a sequence is referred to as a “pulse burst”), followed by a period in which no pulses are generated (the off-time_, and the nerve is allowed to recover or rest from the stimulation provided by the pulse burst. Continuous stimulation modes may be provided by programming the off-time to zero. In conventional non-continuous stimulation, the off-time may range up to one day or more. Typically, however, the ratio of the off-time to on-time ranges from approximately 0.5 to 20, i.e., the off-time is from half as long as the on-time to twenty times the length of the on-time. For example, one suitable pattern of intermittent continuous stimulation may comprise 30 seconds on and five minutes off, for 24 hours a day, 7 days a week. The electrical signal parameters should be adjusted so as to deter or prevent the occurrence of coronary vasoconstriction or spasm in a patient suffering from chronic angina.

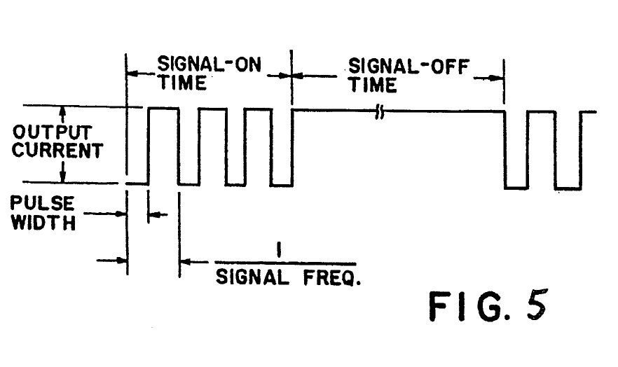

FIG. 5 is a simplified representation of an output signal waveform delivered by the pulse generator 10 to electrode assembly 70, which illustrates the configurable (programmable) parameters of on-time, off-time, frequency, pulse width, and output current for the output signal. Nominally, the width of each pulse is set to a value not greater than about 1 millisecond, more typically 250-500 microseconds, and the pulse repetition frequency may programmed to be in a range of about 20 to 250 Hz. In one embodiment, a frequency of 30 Hz is used. A nonuniform frequency may also prove advantageous in alleviating acute angina and/or in deterring the onset of an acute episode. Frequency may be altered during a pulse burst by either a frequency sweep from a low frequency to a higher frequency or vice versa.

It is contemplated that application of different stages or phases of such therapeutic electrical signals will be especially advantageous for managing angina conditions in patients. A first electrical pulse signal may provide a stimulation signal for a first, acute treatment period, to strongly dilate a coronary artery to ameliorate acute angina. A second electrical pulse signal, different from the first electrical pulse signal, may provide a stimulation signal for a second, chronic treatment period. In one embodiment, the first (acute) electrical pulse signal provides a higher level of stimulation than said second (chronic) electrical pulse signal. In a still further embodiment, the first electrical pulse signal may be triggered by the patient manually, e.g., by a magnet signal, during periods of angina pain. The electrical and timing parameters of the stimulating signal used for vagus nerve stimulation (VNS) as described herein for the preferred treatment regimes are merely exemplary and not as constituting limitations on the scope of the invention, except insofar as recited in the claims.

Also, as shown in FIG. 4B, dual implanted pulse generators 10a and 10b may be used, one coupled to the right vagus and the other to the left vagus to provide the bilateral stimulation. While the pulse generators 10a and 10b may be programmed with the same stimulation parameters, different stimulation parameters may also be effective in producing artery dilation for treating angina. Use of implanted stimulators is preferred; however, treatment may instead be administered using an external stimulator with an internally implanted lead and electrode that are inductively coupled to the external stimulator, as illustrated in FIG. 2 of U.S. Pat. No. 4,867,164. Wholly external stimulation may also be used on an out-patient basis, although implantation of at least the electrode and the lead is preferred for a more efficient electrical coupling to the nerve. Moreover, implantation of one or more neurostimulators advantageously allows the patient to be completely ambulatory during treatment, so that normal daily routine activities are unaffected.

Still another stimulation regimen includes programming the output of the pulse generator to the maximum amplitude that the patient can tolerate, and programming the pulse generator to cycle the electrical signal on and off for a predetermined period of time, followed by a relatively long interval without stimulation.

In some embodiments, sensors may be employed to provide feedback loop stimulation. The most common form of feedback loop stimulation is manually triggered stimulation, in which the patient manually causes the activation of a stimulation pulse burst outside of the programmed on-time/off-time cycle. For example, if the patient senses the onset of angina, he or she may manually activate the neurostimulator to stimulate the predetermined location on the vagus nerve, thus eliminating or substantially reducing the level of pain. The parameter settings for manually triggered stimulation may provide a higher level of stimulation than passive stimulation, and thus may constitute an acute phase treatment parameter set as discussed above. The patient also may be allowed to alter the intensity of the signals applied to the vagus nerve within prescribed ranges. For example, the patient may alter the signal frequency, current, duty cycle or a combination thereof In at least some treatment regimes, the neurostimulator is programmed to generate the stimulus for a relatively long period of time in response to manual activation. In this way, treatment is designed, in part, to increase the activity of the vagus nerve is such a way that arterial constriction is avoided or deterred so that the onset of angina pectoris is also avoided, or the frequency and/or intensity of angina episodes is reduced.

Patient activation of the neurostimulator may involve use of an external control magnet for operating a reed switch in the implanted device, for example. Certain other applicable techniques of manual and automatic activation of implantable medical devices are disclosed in U.S. Pat. No. 5,304,206 (Cyberonics, Inc.). For example, means for manually activating or deactivating the stimulus generator may include a sensor such as a piezoelectric element mounted to the inner surface of the generator case 27 and adapted to detect light taps by the patient on the implant site. One or more taps applied in a predetermined sequence to the skin above the location of the stimulus generator 10 in the patient's body may be programmed into the device as the signal for activation of the generator, whereas a different sequence of taps spaced apart may be programmed as the signal for deactivation, for example. The therapy regimen performed by the implanted device(s) remains that which has been pre-programmed by means of the external programmer, according to the prescription of the patient's physician. In this way, the patient is given limited but convenient control over the device operation, to an extent which is determined by the program dictated and/or entered by the attending physician. The patient also may activate the neurostimulator using other suitable techniques and/or apparatus. In this mode of operation a steady state stimulation pattern is established, with the patient being able to intermittently supplement or boost the level of stimulation within a prescribed range, according to the perceived symptoms of angina.

Feedback-enhanced Stimulation. Reference is now made to the feedback-enhanced configuration which is also shown in FIG. 1, with the sensor 140 and lead 130 depicted in dashed lines. In instances in which physiological feedback-enhanced stimulation is desired, without manually triggering by the patient, sensor 140 preferably senses a body parameter that corresponds to a symptom or physical indication of ischemia or angina. If sense electrodes are to be utilized to detect early indicators or onset of angina (e.g., manifested by continuous or intermittent mild chest pain, or decreased blood flow in a coronary artery), a signal analysis circuit is preferably included in the electrical pulse generator 10 for processing and analyzing the signals from the sensor 140. Upon detection of one or more selected angina indicator, the processed signal is supplied to a microprocessor to trigger delivery of the electrical signal to the selected effector site on the nerve. In one contemplated variation of this feedback-enhanced treatment regime, the detection of an angina indicator may be used to trigger a stimulation program comprising different stimulation parameters from a passive stimulation program, such as having a higher current or a higher ratio of on-time to off-time.

The above-described methods are believed to be especially useful to physicians in formulating appropriate therapeutic treatment for dilation of at least a portion of a coronary artery of patients having chronic stable angina conditions. With appropriate precautions and patient cooperation, these methods may also play a useful role in alleviating pain in patients suffering from chronic unstable angina conditions.

The foregoing embodiments are to be construed as illustrative, and not as constraining the remainder of the disclosure. Many variations and modifications of the embodiments disclosed herein are possible and are within the scope of the invention. For instance, the above-described principles of operation may be applicable to selected cranial nerves other than the vagus nerve, to achieve the desired results by coupling one or more stimulation electrode to one or more respective effector site. Hence, although certain preferred methods and modes of treating and controlling angina through a regimen of cranial nerve, especially vagus nerve, stimulation directly or indirectly at predetermined effector locations or sites have been described herein, it will be appreciated that variations and modifications may be made within the scope of the present invention as defined by the appended claims. Accordingly, the scope of protection is not limited by the description set out above, but is only limited by the claims which follow, that scope including all equivalents of the subject matter of the claims. The disclosures of all patents, patent applications and publications cited herein are hereby incorporated herein by reference, to the extent that they provide exemplary, procedural or other detailssupplementary to those set forth herein.

References.

The following publications are cited bynumber in the foregoing text:

1. Braunwald, Eugene et al., “Relief of Angina Pectoris by Electrical Stimulation of the Carotid-Sinus Nerves” New England Journal of Medicine 277(24) 1278-1283 Dec. 14, 1967).

2. Epstein, Stephen E. et al., “Treatment of Angina Pectoris by Electrical Stimulation of the Carotid-sinus Nerves” New England Journal of Medicine 280(18) 971-978 (May 1, 1969).

3. Zefi, Howard J. et al., Letter to the Editor, New England Journal of Medicine 281(2) 103 (Jul. 10, 1969).

4. Epstein, Stephen E., Letter to the Editor, New England Journal of Medicine 281(2) 104 (Jul. 10, 1969).

5. Epstein, Stephen E., et al., “Circulatory Effects of Electrical Stimulation of the Carotid Sinus Nerves in Man” Circulation XL(3):269-276 (September 1969).

6. Braunwald, Nina S. et al., “Carotid Sinus Nerve Stimulation for the Treatment of Intractable Angina Pectoris: Surgical Technique” Annals of Surgery 172(5) 870-876 (November 1970).

7. Cohn, Keith, Letter to the Editor, Circulation XLIX: 1015-1016 (May 1974).

8. Goldstein, Robert E. and Stephen E. Epstein, Letter to the Editor, Circulation XLIX: 1017-(May 1974).

9. Chandler, Margaret J. et al., “Vagal Sympathetic and Somatic Sensory Inputs to Upper Cervical (C1-C3) Spinothalamic Tract Neurons in Monkeys” Journal of Neurophysiology 76(4):2555-2567 (October 1996).

What is claimed is:

1. A method of treating angina pectoris in a patient, the method comprising:
applying a therapeutic electrical signal to an electrode coupled to a cranial nerve of the patient at a selected site on the nerve, the signal being effective to dilate at least a portion of a coronary artery of the patient and thereby alleviate the angina without significantly slowing the heart rate ofthe patient.

2. The method of claim 2 wherein said electrode is coupled to a vagus nerve of the patient.

3. The method of claim 2 wherein said vagus nerve is a left vagus nerve of the patient.

4. The method of claim 2 comprising:
providing a pulse generator capable of generating a pulsed electrical signal;
providing at least one electrode;
implanting the pulse generator in the patient's body;
surgically coupling said at least one electrode directly to a vagus nerve of the patient at a selected site on said nerve that is capable of causing dilation of at least a portion of a coronary artery of the patient when electrical stimulation is applied to said site;
coupling the pulse generator to the electrode;
generating a predetermined pulsed electrical signal by said pulse generator; and
applying said pulsed electrical signal to said at least one electrode such that at least a portion of said artery dilates and thereby at least partially relieves angina pectoris in said patient.

5. The method of claim 4 further comprising programming said electrical pulse generator to define said pulsed electrical signal by a plurality of predetermined parameters including a current magnitude, a pulse frequency, and a pulse width, wherein said predetermined parameters are capable of causing dilation of at least a portion of a coronary artery of the patient when said pulsed electrical signal is applied to said at least one electrode.

6. The method of claim 4 further comprising:
detecting at least one indicator of the onset of angina pectoris; and
applying of said electrical signal to said vagus nerve in response to the detection of said at least one indicator.

7. The method of claim 6 wherein said step of detecting comprises the patient subjectively experiencing chest pain, and said step of initiating comprises the patient taking action to start said application of said electrical signal.

8. The method of claim 6 further comprising:
obtaining an implantable sensor that is capable of sensing said indicator of the onset of angina pectoris;
implanting the sensor in the patient's body at a predetermined sensing site; and
coupling the sensor to the programmed pulse generator, wherein said detecting comprises sensing said indicator of the onset of angina pectoris with said sensor.

9. The method of claim 8 wherein said at least one indicator of the onset of angina comprises decreased blood flow to the heart, and said sensor comprises a blood pressure sensor for measuring blood pressure.

10. The method of claim 6 wherein said at least one therapeutic electrical signal comprises:
a first therapeutic electrical signal for treating acute angina, wherein said first signal comprises a programmed first level of stimulation;
a second therapeutic electrical signal for treating chronic angina, wherein said second signal comprises a different level of stimulation than said first level of stimulation.

11. The method of claim 10 wherein said step of applying said at least one therapeutic signal comprises:
applying said second signal to said electrode; and
intermittently interrupting said application of said second signal with application of said first signal upon detecting said at least one indicator of the onset of angina pectoris.

12. The method of claim 11 wherein said step of intermittently interrupting application of said second signal with application of said first signal comprises sustaining application of said first signal until alleviation of angina is obtained or until said at least one indicator ceases to be detected.

13. The method of claim 12 wherein said patient suffers from chronic angina and said step of sustainedly applying said first therapeutic electrical signal is effective to dilate, or deter vasoconstriction of, at least a portion of a coronary artery of the patient and thereby deter or avoid onset of acute angina.

14. The method of claim 1 wherein said step of coupling said electrode at said selected site on said nerve comprises positioning said electrode on the left vagus nerve in the neck area below the cardiac branch ofthe vagus nerve.

15. The method of claim 1 further comprising administering a pharmaceutical agent to said patient to further alleviate angina.

16. The method of claim 1 wherein said therapeutic electrical signal comprises an on-time and an off-time, and wherein said on-time comprises a time within the range of 1 second to 1 day, and said off-time comprises a time within the range of 0 seconds to 1 day.

17. A method of selectively dilating a coronary artery in a patient in need thereof, the method comprising:
applying at least one therapeutic electrical signal to an electrode coupled to a cranial nerve of the patient at a selected site on said nerve effective to dilate at least a portion of the coronary artery and thereby increasing blood flow to the patient's heart such that the increased arterial blood flow alleviates angina without significantly slowing the patients heart rate.

18. The method of claim 17 wherein said at least one therapeutic electrical signal is effective to deter the onset of an episode of acute angina.

19. The method of claim 17 comprising selecting a site on said nerve that has been determined to be a coronary artery-dilation effector site, wherein, when said effector site is stimulated at least a portion of a coronary artery is caused to be dilated inresponse to such stimulation.

20. The method of claim 17 wherein said cranial nerve is the vagus nerve of the patient and said selected site is an area on said nerve below the cardiac branches of the vagus nerve.

21. The method of claim 17 further comprising monitoring the heart rate of said patient, wherein said step of applying said at least one therapeutic electrical signal does not cause the patient's heart rate to decrease more than about 20%.

22. The method of claim 21 wherein said step of applying said at least one therapeutic electrical signal does not cause the patients heart rate to decrease more than about 10%.

23. An implantable apparatus for deterring or preventing vasoconstriction or spasm of a coronary artery in a patient in need thereof the apparatus comprising:
an electrode coupled to a cranial nerve of the patient to stimulate said cranial nerve;
a power supply;
a pulse generator coupled to said power supply and to said electrode, wherein said pulse generator is adapted to apply a therapeutic electrical signal to said cranial nerve using said electrode, wherein said electrical signal is effective to modulate neuronal activity of said cranial nerve to counteract constriction of at least a portion of the artery without significantly slowing the patient's heart rate.
